(19) United States
(12) Patent Application Publication
(10) Pub. No.: US 2026/0183559 A1
(43) Pub. Date: Jul. 2, 2026

(54) RED-LIGHT THERAPY (RLT) DEVICE

(71) Applicant: Joseph John Terndrup, Fort Worth, TX (US)

(72) Inventor: Joseph John Terndrup, Fort Worth, TX (US)

(73) Assignee: HoldingCo LLC, Burleson, TX (US)

(21) Appl. No.: 19/001,600

(22) Filed: Dec. 26, 2024

(51) Int. Cl. A61N 5/06; A61B 17/00; A61C 5/90

(52) U.S. Cl. CPC A61N 5/0601; A61C 5/90; A61B 2017/00734; A61N 2005/0606; A61N 2005/0626; A61N 2005/0663

(57) ABSTRACT

A hands-free, Red-Light Therapy device comprising a first base that stays outside the mouth and a semicircular or U-shaped lip spacer inserted into the mouth, with extended arms featuring inner tooth spacers. It has three sets of LEDs: a node on an adjustable, flexible, and detachable arm attached to the lip spacer, LEDs around the outer surface of the lip spacer, and LEDs on the side wall of the first base. Powered by a rechargeable battery with a timer switch, the device delivers red light (633-850 nm) to the inner mouth, lips, gums, and teeth, facilitating the treatment of mouth sores and injuries. The lip spacer is designed to open and hold the mouth securely using tooth spacers sized to fit 2-3 molars. This invention addresses the need for a convenient and effective at-home treatment for oral pain and sores, combining the benefits of red-light therapy with hands-free operation.

### TECHNICAL FIELD

The present invention relates to the field of devices for Light-Based Therapies that utilize specific wavelengths of light for therapeutic purposes, including the treatment of skin conditions, pain management, and promoting healing.

### BACKGROUND

Red Light Therapy (RLT), also known as photobiomodulation or low-level light therapy, is a non-invasive treatment that uses specific wavelengths of red and near-infrared light to penetrate the skin and promote cellular regeneration and healing. This non-invasive treatment has been used for various medical and cosmetic applications, such as reducing inflammation, accelerating wound healing, and alleviating pain. The therapeutic benefits of RLT are attributed to its ability to stimulate mitochondrial activity, leading to increased ATP production and enhanced cellular repair processes.

Oral health is a critical aspect of overall well-being, and many individuals suffer from common oral issues such as mouth sores, ulcers, and other sources of oral pain. These conditions can be caused by various factors, including infections, injuries, and inflammatory diseases. Traditional treatments for oral pain and sores often involve the use of topical medications, mouth rinses, and other palliative measures. However, these treatments may not provide sufficient relief or expedite the healing process.

Handheld Red Light Therapy Devices: Handheld RLT devices have been available for some time and are commonly used for treating various skin conditions and promoting healing. These devices typically require the user to hold the device in place, which can be inconvenient and impractical, especially for applications within the oral cavity.

Topical Applications and Mouth Rinses: For oral health issues, topical treatments and mouth rinses containing antiseptic, anti-inflammatory, or anesthetic agents have been widely used. While these methods can provide temporary relief, they do not offer the regenerative benefits of RLT and may not accelerate the healing process effectively.

Dental Lasers: Dental professionals have used laser devices to treat various oral health conditions. These lasers can be effective but are typically used in a clinical setting due to their cost and the expertise required for operation. They are not designed for convenient, at-home use by patients.

Fixed RLT Panels and Masks: Fixed RLT panels and masks have been developed for hands-free treatment of larger areas of the body, primarily for skin rejuvenation and pain relief. These devices, however, are not specifically designed for targeted treatment of the oral cavity and may not be effective for addressing localized oral health issues.

Despite the availability of various RLT devices and treatments for oral health issues, there has been a lack of hands-free, medical-grade RLT devices specifically designed for the mouth, lips, and oral cavity. This gap in the market means that patients with oral pain and sores have limited options for convenient and effective at-home treatment. The need for a device that combines the proven benefits of red-light therapy with the practicality of hands-free operation is evident.

The above information disclosed in this background section is only for enhancement of understanding of the background of the inventive concept, and, therefore, it may contain information that does not form the prior art that is already known to a person of ordinary skill in the art.

### SUMMARY

The invention discloses a Red-Light Therapy (RLT) device for oral cavity including mouth and lip treatment that includes a first base that remains outside the mouth and a semicircular or U-shaped lip spacer, which is inserted into the mouth. The lip spacer has two extended arms with inner tooth spacers, and it is attached to the first base. The device features three sets of LEDs: the first set is a node attached to an adjustable, flexible, and detachable arm connected to the lip spacer, the second set surrounds the outer surface of the lip spacer, and the third set is on the side wall of the first base. The device is powered by a rechargeable battery housed in the first base, which also includes a timer switch for preset therapy durations. This hands-free device delivers red light (633-850 nm) therapy to the inner mouth, lips, gums, and teeth, facilitating the treatment of mouth sores and injuries. The lip spacer is designed to open and hold the mouth in place, with tooth spacers sized to fit 2-3 molars, ensuring the device is securely held.

The invention addresses the gap in the market by providing a hands-free, medical-grade RLT device specifically designed for the mouth, lips, and oral cavity. The device targets multiple locations within the mouth, including the outer and inner lips, gums, tongue, and mouth. It uses RLT technology to speed up the healing of mouth sores and provides additional benefits such as pain relief, anti-scarring effects, and overall oral and gum health improvement.

### BRIEF DESCRIPTION OF THE DRAWINGS

The accompanying drawings, which are included to provide a further understanding of the inventive concepts and are incorporated in and constitute a part of this specification, illustrate exemplary embodiments of the inventive concepts, and, together with the description, explain the principles of the inventive concepts.

FIG. 1 shows the front isometric view of a preferred embodiment of the invention.

FIG. 2 shows the back isometric view of the preferred embodiment of the invention.

FIG. 3 shows the exploded view of the preferred embodiment of the invention.

FIG. 4 shows the front view of the preferred embodiment of the invention.

FIG. 5 shows the back view of the preferred embodiment of the invention.

FIG. 6 shows the top view of the preferred embodiment of the invention.

FIG. 7 shows the side view of the preferred embodiment of the invention.

FIG. 8 shows the front isometric view of another embodiment of the invention.

FIG. 9 shows the back isometric view of the embodiment of FIG. 8.

FIG. 10 shows the exploded view of the embodiment of FIG. 8.

FIG. 11 shows the front view of the embodiment of FIG. 8.

FIG. 12 shows the back view of the embodiment of FIG. 8.

FIG. 13 shows the top view of the embodiment of FIG. 8.

FIG. 14 shows the side view of the embodiment of FIG. 8.

### DESCRIPTION OF THE INVENTION

In the following description, for the purposes of explanation, numerous specific details are set forth to provide a thorough understanding of various exemplary embodiments. It is apparent, however, that various exemplary embodiments may be practiced without these specific details or with one or more equivalent arrangements.

In the accompanying figures, the size and relative sizes of elements may be exaggerated for clarity and descriptive purposes.

The terminology used herein is for the purpose of describing embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof.

The present invention discloses a Light Therapy (LT) device, to deliver light to oral cavity specifically to inner lip & gums, outer lips & corner of lips, and Inner mouth comprising gums, tongue, hard palette, soft palette, and surrounding areas. The device is a hands-free device comprising a first base 2, a lip spacer 1, two tooth spacers, three LT applicators, and a power source.

The lip spacer 1 is a semicircular or U-shaped structure designed to be inserted into the mouth. It may be of any other shape convenient to fit into the mouth. It comprises a second base having two extended arms at its one end and attached to the first base 2 at its other end, wherein the first base 2 is broader in width than the second base. It is designed to adjust the size of the mouth. It is designed to open the mouth and hold the device in place by pressing the tooth spacers by teeth and allowing the lips to rest on the second base.

There are two tooth spacers, one attached to the inner surface of each of the extended arms of the lip spacer 1. The tooth spacers are of the size of 2-3 molars and allow the molars to hold the device in place while also spacing the teeth wide enough for the arm to run between them.

The tooth spacers may be made up of poly (ethylene vinyl acetate-EVA) copolymer, gel or moldable plastic and may be detachable. This feature allows the user to take the part that contacts the teeth and mouth and place it in hot water and then bite down on it for a few seconds allowing it to custom mold to his mouth. This is a comfortable-fit feature of the device where the teeth bite down to provide a more comfortable bite and a better grip for the teeth.

The first LT applicator comprises a first set of LEDs in the form of a node attached to an arm 3 that is further attached to the lip spacer 1. The arm 3 may be attached to the lip spacer 1 at the middle of its cavity through a magnet or any other means. The arm 3 is adjustable, flexible and may be fixed or detachable. It delivers light to the inner mouth, rear gums, tongue, uvula, and palate.

The second LT applicator 6 comprises a second set of LEDs that may be present around the outer surface of the lip spacer 1, along the circumference of the second base and, optionally, on the outer sides of the extended arms as well. The second applicator 6 delivers light to the inner lip, inner cheeks, and front gums.

The third LT applicator 4 comprises a third set of LEDs that may be present on sidewall of the first base along its circumference and may be perpendicular to angle of the second set of LEDs. The third applicator delivers light to the outer lip and outside of the mouth.

The power source may be a rechargeable battery 7 connected to a power switch 5. The first base of the device remains outside the mouth and is designed to house the battery 7, a charging port, an optional timer switch, a power switch 5 and control buttons. The timer switch may be connected to the power switch 5 and allows predetermined therapy time frames.

The Light Therapy delivered by the device may be Red-Light Therapy (RLT) wherein the LEDs deliver red light. The frequency of the red light may range from 633 nm to 850 nm. The RLT may be used in the therapy of the inner mouth, inner lips, outer lips, gums and teeth to treat the mouth sores and injuries by delivering red light to them.

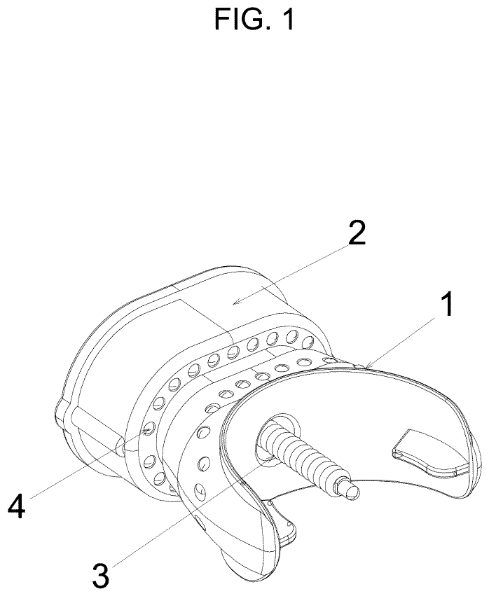

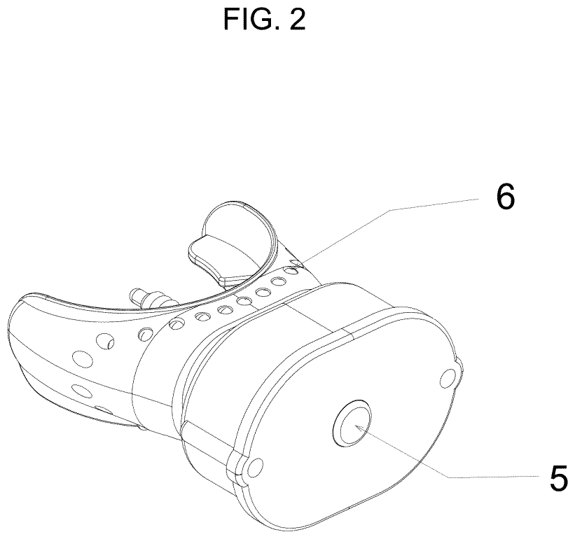

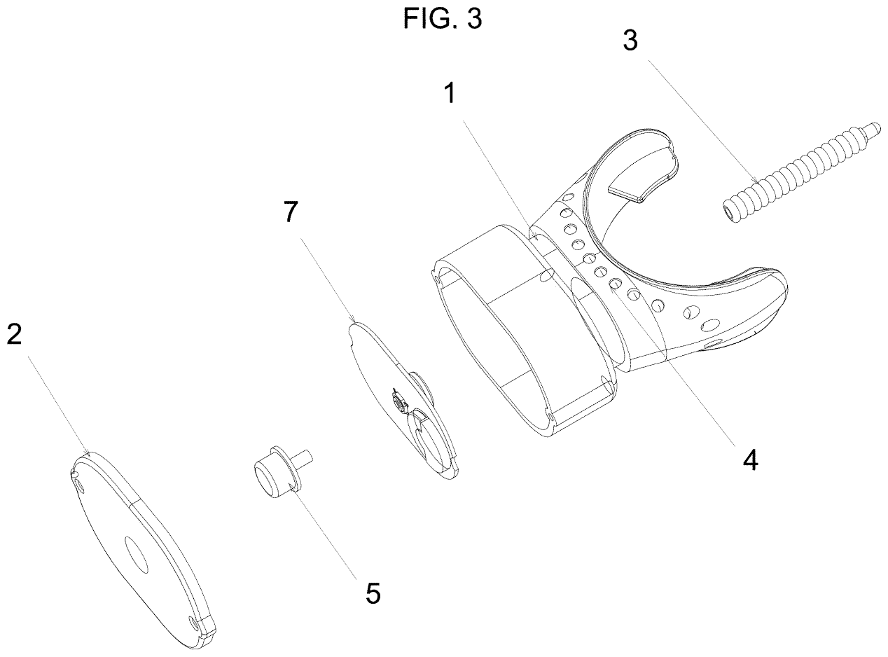

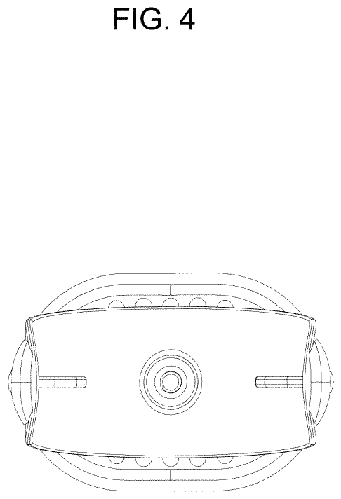

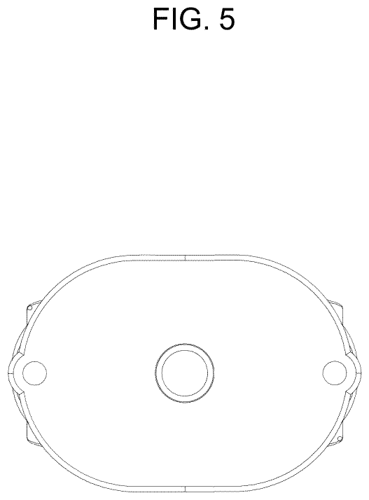

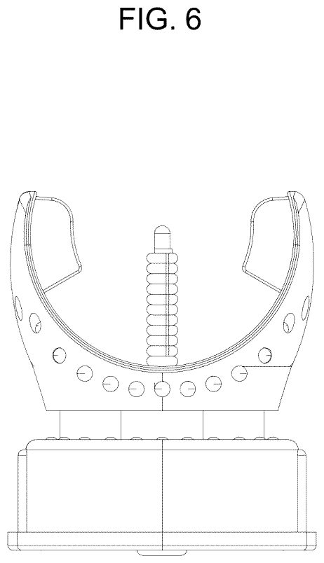

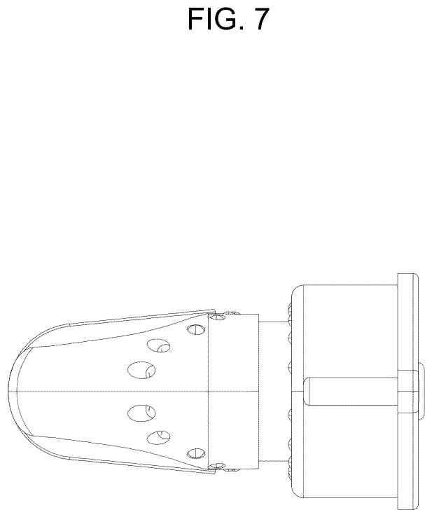

A preferred embodiment of the invention, FIG. 1 to FIG. 7, discloses a hands-free, three-in-one, Red Light Therapy (RLT) device, to deliver red light to oral cavity specifically to inner lip & gums, outer lips & corner of lips, and Inner mouth comprising gums, tongue, hard palette, soft palette, and surrounding areas, comprising:
a first base 2, to remain outside the mouth;
a lip spacer 1, to be inserted into the mouth, comprising a second base having two extended arms at its one end and attached to the first base at its other end, wherein the first base is broader in width than the second base;
two tooth spacers, one on the inner surface of each of the extended arms;
a first RLT applicator comprising a first set of LEDs in the form of a node attached to an arm 3 that is further attached to the lip spacer 1;
a second RLT applicator 6 comprising a second set of LEDs present around the outer surface of the lip spacer 1, along the circumference of the second base and the outer sides of the extended arms;
a third RLT applicator 4 comprising a third set of LEDs present on side-wall of the first base along its circumference and perpendicular to angle of the second set of LEDs; and
a power source for the device.

The arm 3 of the first applicator of the preferred embodiment is adjustable, flexible and detachable, and is attached to the lip spacer 1 the middle of its cavity through a magnet. The lip spacer 1 is semicircular or U-shaped structure and is designed to adjust the size of the mouth. It is designed to open the mouth and hold the device in place by pressing the tooth spacers by teeth and allowing the lips to rest on the second base.

The first applicator of the preferred embodiment delivers red light to the inner mouth, rear gums, tongue, uvula, and palate. Whereas the second applicator 6 delivers red light to the inner lip, inner cheeks, and front gums. The third applicator delivers red light to the outer lip and outside of the mouth.

The tooth spacers of the preferred embodiment are of the size of 2-3 molars and allow the molars to hold the device in place while also spacing the teeth wide enough for the arm 3 to run between them. The tooth spacers are made up of poly (ethylene vinyl acetate-EVA) copolymer and are detachable, this allows the user to take the part that contacts the teeth and mouth and place it in hot water and then bite down on it for a few seconds allowing it to custom mold to his mouth.

The preferred embodiment delivers red light of the frequency of 633 nm to 850 nm. The device is used in the therapy of the inner mouth, inner lips, outer lips, gums and teeth to treat the mouth sores and injuries by delivering red light to them.

The preferred embodiment further comprises a rechargeable battery 7 connected to a power switch 5 to turn it on and off. There is a timer switch, connected to the power switch 5, that allows predetermined therapy time frames. The first base 2 of the embodiment remains outside the mouth and houses the battery 7, charging port, the timer switch, the power switch 5 and control buttons.

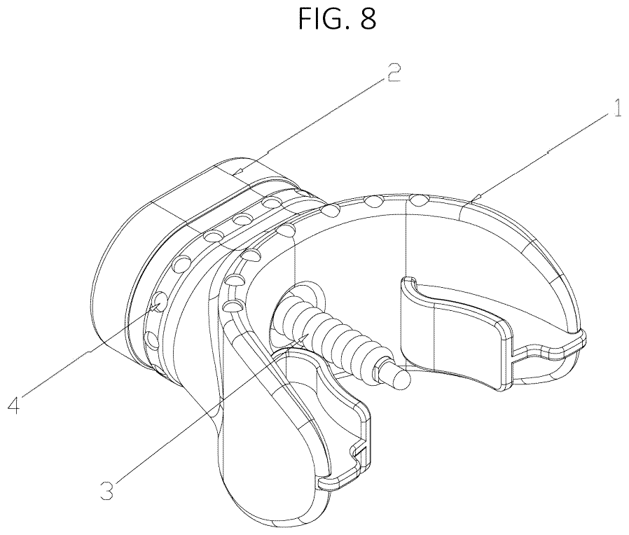

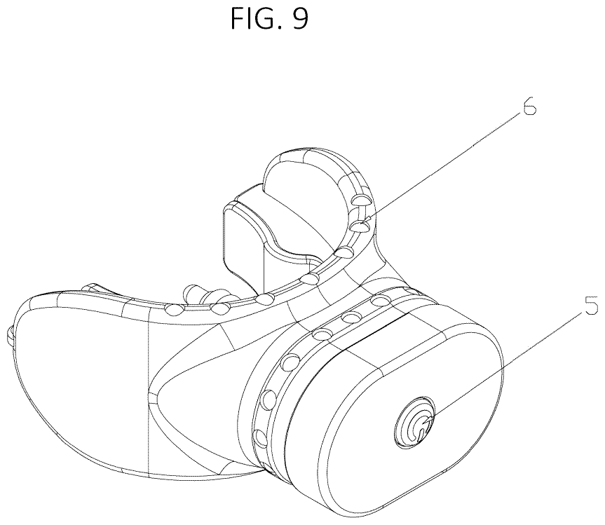

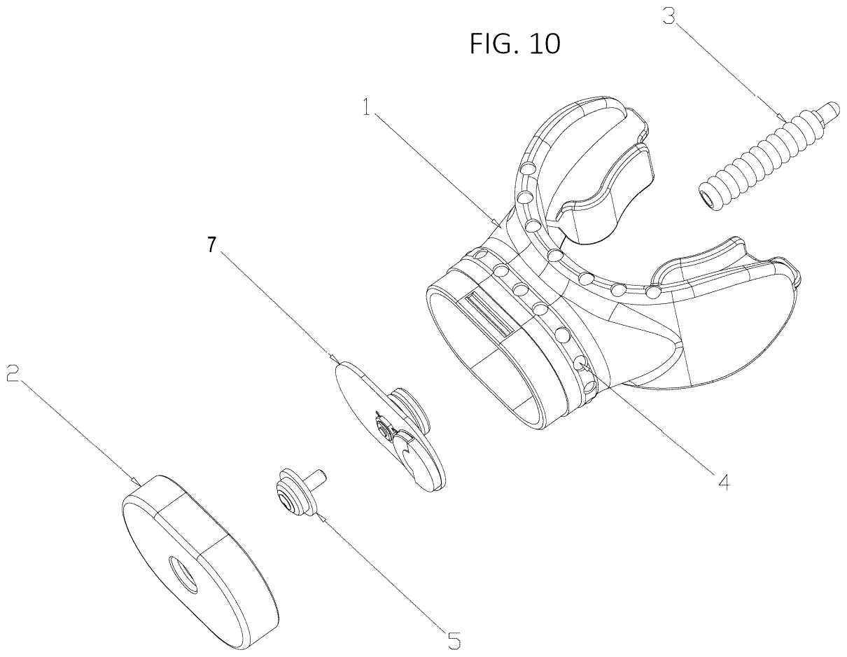

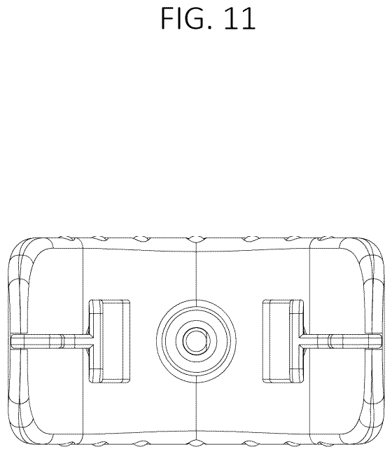

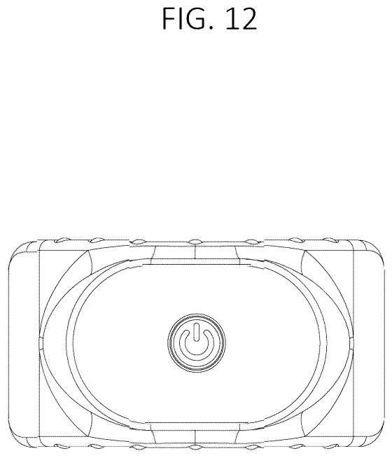

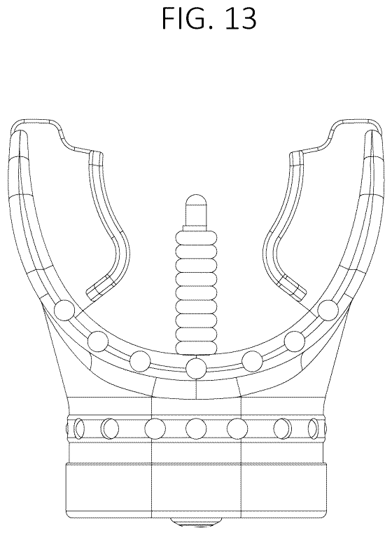

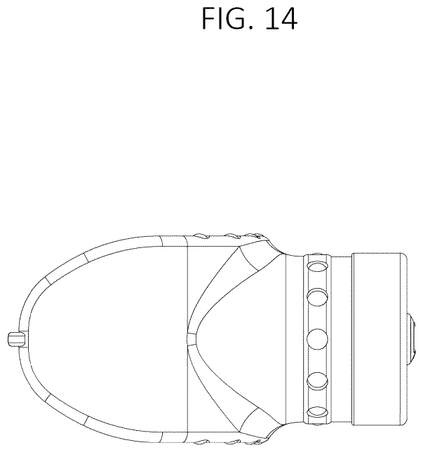

FIG. 8 to FIG. 14 disclose another preferred embodiment of the invention wherein the second RLT applicator 6 comprises a second set of LEDs present around the outer top surface of the lip spacer 1. The third RLT applicator 4 comprises a third set of LEDs present on outer top surface of the first base along its circumference and parallel to angle of the second set of LEDs.

The invention further discloses a method of treatment of mouth sores and injuries by exposing them to red light. The frequency of red light may range from 633 nm to 850 nm.

The invention provides a hands-free Light Therapy (LT) device designed explicitly for use in the mouth, lips, and oral cavity. This device allows users to receive the therapeutic benefits of light therapy in a convenient and comfortable manner, promoting faster recovery and healing of common mouth sores and oral pain. The device is designed to be user-friendly and suitable for at-home use, making it accessible to a broader range of individuals seeking effective treatment for their oral health issues.

### Benefits of the Invention

360-Degree Outer Lip Applicator: Provides comprehensive red light therapy coverage to the outer lip area.
360-Degree Inner Lip Applicator: Ensures thorough treatment of the inner lip region.
Removable Inner Mouth Light: Attaches via a magnet and is flexible, allowing targeted treatment of specific areas such as cold or canker sores without requiring the user to hold the light.
The device is held in place via a rear tooth spacer and a front lip rest, ensuring comfort and stability during use. The design of the device allows for hands-free application of red-light therapy, making it convenient for users to receive treatment while engaging in other activities.
Accelerates Recovery: Speeds up the healing of mouth sores and oral injuries.
Pain Relief: Provides relief from pain associated with oral conditions.
Anti-Scarring: Reduces the visibility of scars, particularly around the mouth area.
Skin Rejuvenation: Promotes overall skin health and rejuvenation.
Hands-Free Operation: Allows for convenient and effective treatment without the need for manual operation.

The invention offers a practical and effective solution for addressing common oral health issues, leveraging the proven benefits of red-light therapy in a user-friendly, hands-free device.

What is claimed is

1. A Light Therapy (LT) device, to deliver light to oral cavity, comprising:
a first base, to remain outside the mouth;
a lip spacer, to be inserted into the mouth, comprising a second base having two extended arms at its one end and attached to the first base at its other end, wherein the first base is broader in width than the second base;
two tooth spacers, one on the inner surface of each of the extended arms;
a first LT applicator comprising a first set of LEDs in the form of a node attached to an arm that is further attached to the lip spacer;
a second LT applicator comprising a second set of LEDs present around the outer surface of the lip spacer, along the circumference of the second base and the outer sides of the extended arms;
a third LT applicator comprising a third set of LEDs present on sidewall of the first base along its circumference and perpendicular to angle of the second set of LEDs; and
a power source for the device.

2. The device of claim 1, wherein the lip spacer is semicircular or U-shaped in shape.

3. The device of claim 1, wherein the arm of the first applicator is attached to the lip spacer at the middle of its cavity.

4. The device of claim 1, wherein the arm is adjustable, flexible and detachable.

5. The device of claim 1, wherein the arm is attached to the lip spacer through a magnet.

6. The device of claim 1, wherein the power source is a rechargeable battery connected to a power switch.

7. The device of claim 1 wherein there is a timer switch, connected to the power switch, that allows predetermined therapy time frames.

8. The device of claim 1, wherein the first base remains outside the mouth and houses the battery, charging port, the timer switch, the power switch and control buttons.

9. The device of claim 1, wherein the first applicator delivers light to the inner mouth, rear gums, tongue, uvula, and palate.

10. The device of claim 1, wherein the second applicator delivers light to the inner lip, inner cheeks, and front gums.

11. The device of claim 1, wherein the third applicator delivers light to the outer lip and outside of the mouth.

12. The device of claim 1, which is a hands-free device.

13. The device of claim 1, wherein the lip spacer is designed to adjust the size of the mouth.

14. The device of claim 1, wherein the lip spacer is designed to open the mouth and hold the device in place by pressing the tooth spacers by teeth and allowing the lips to rest on the second base.

15. The device of claim 1, wherein the tooth spacers are of the size of 2-3 molars and allow the molars to hold the device in place while also spacing the teeth wide enough for the arm to run between them.

16. The device of claim 1, wherein the tooth spacers are made up of poly (ethylene vinyl acetate-EVA) copolymer and are detachable, this allows the user to take that part and place it in hot water and then bite down on it for a few seconds allowing it to custom mold to his mouth.

17. The device of claim 1, wherein the Light Therapy is Red Light Therapy (RLT) and the LEDs deliver red light.

18. The device of claim 17, wherein the frequency of red light is from 633 nm to 850 nm.

19. The device of claim 1, for use in the therapy of the inner mouth, inner lips, outer lips, gums.

20. The device of claim 1, wherein the second RLT applicator comprises a second set of LEDs present around the outer top surface of the lip spacer, and wherein the third RLT applicator comprises a third set of LEDs present on outer top surface of the first base along its circumference and parallel to angle of the second set of LEDs.

21. A method of treatment of mouth sores and injuries by exposing them to red light of frequency of 633 nm to 850 nm.
